(19) United States
(12) Patent Application Publication
(10) Pub. No.: US 2013/0023587 A1
(43) Pub. Date: Jan. 24, 2013

(54) COMPOSITIONS AND METHODS FOR THE TREATMENT OF CANCER

(71) Applicants: Thies Schroeder, Durham, NC (US); Mark W. Dewhirst, Durham, NC (US)

(72) Inventors: Thies Schroeder, Durham, NC (US); Mark W. Dewhirst, Durham, NC (US)

(73) Assignee: DUKE UNIVERSITY, Durham, NC (US)

(21) Appl. No.: 13/638,858

(22) PCT Filed: Apr. 1, 2011
(86) PCT No.: PCT/US11/30953

§ 371 Date: Oct. 1, 2012

Related U.S. Application Data
(60) Provisional application No. 61/320,093, filed on Apr. 1, 2010.
(60) Provisional application No. 61/341,860, filed on Apr. 6, 2010.

(51) Int. Cl. C12N 5/09; A61P 35/00; A61K 31/277

(52) U.S. Cl. 514/521; 435/375

(57) ABSTRACT

Provided are methods for inhibiting survival of a proliferating, quiescent, or hypoxic cancer cell that include administering a compound that inhibits lactate clearance (e.g., lactate catabolism, lactate transport, glutamate release, and/or alanine release) in the proliferating, quiescent, or hypoxic cancer cell. Further provided are methods of reducing the proliferation of a neoplastic cell having increased lactate or decreased glucose concentrations relative to a normal cell. Also provided are methods of treating a subject having cancer, wherein the methods may include administering to the subject an inhibitor of lactate clearance.

### CROSS-REFERENCE TO RELATED APPLICATIONS

This application claims priority to U.S. Provisional Patent Application No. 61/320,093 filed Apr. 1, 2010, and U.S. Provisional Patent Application No. 61/341,860 filed Apr. 6, 2010, each of which is incorporated herein by reference in its entirety.

### STATEMENT REGARDING FEDERALLY-SPONSORED RESEARCH

This invention was made with government support under grant number CA40355 awarded by the National Cancer Institute. The United States government has certain rights in the invention.

### FIELD

The disclosure relates generally to active agents and associated methods for treating cancer, inhibiting or reducing proliferation, size, and/or mass of a solid tumor, and inhibiting lactate clearance in a tumor cell.

### BACKGROUND

Traditional chemotherapy targets proliferating cells, based on the assumption that uncontrolled proliferation is what separates cancer from normal tissue. However, a considerable percentage (approx. 30-80%) of cancer cells in solid tumors do not actually proliferate, but stay quiescent. Cancer cell growth arrest (quiescence) is caused by factors associated with temporary or permanent distance from supplying vasculature, such as hypoxia, nutrient starvation, deprivation of growth factors, and build-up of toxic waste. Quiescent cancer cells have the ability to repopulate a tumor, after proliferating cancer cells are eliminated by cycles of cytotoxic therapy. In fact, the percentage of quiescent cells is a negative prognostic factor for outcome from therapy in some cancers.

Rather than with growth and anabolic processes, quiescent cancer cells have to be concerned with protecting against the adverse factors associated with their location at distance from the supply lines, such as glucose and oxygen deprivation, reductive stress, extracellular acidity, and build-up of toxic waste. One such factors that can be found in literally all solid tumors is concentrated lactic acid, which inevitably arises from cancer cell glycolysis. Because of poor clearance, lactate in solid tumors builds up to steady state levels of over 40 mM. Although anaerobic exercise can lead to a temporary buildup of lactate in healthy tissues, the degree and persistence of lactate build-up is highly specific to solid tumors. High environmental lactate exerts toxicity to cancer cells, such as reductive stress, intracellular acidification, and inhibition of glycolysis. Quiescent cancer cells in solid tumors are resistant to many conventional therapies such as radiotherapy and chemotherapy. Consequently, quiescent cancer cells are often associated with disease relapse and negative prognosis.

Most cytotoxic anticancer therapies target cells that are dividing, growing, or otherwise proliferating. This class of anticancer therapeutics include alkylating agents, intercalating drugs, and microtubule inhibitors such as taxanes, as well as therapies that target cancer signaling (growth) pathways such as Trastuzumab (targeting Erb2), Cetuximab (Erb1), or Imatinib (Bcr-Abl). Few chemotherapeutic approaches (e.g., anti-angiogenic therapy and hypoxic cytotoxins) target cancer cells by mechanisms other than growth and proliferation. Strategies that target specific cytoprotective mechanisms, such as to block pH regulation in cancer, are even scarcer, and are at early experimental stages. Thus, there is a need to develop therapeutic approaches that target quiescent cancer cells, in addition to antiproliferative therapy, in the treatment of cancer.

### SUMMARY

In an aspect, the disclosure relates to a method of inhibiting survival or growth of a proliferating, quiescent, or hypoxic cancer cell comprising contacting the cancer cell with an agent in an amount effective to inhibit a lactate catabolic pathway in the cancer cell.

In another aspect, the disclosure relates to a method of inhibiting survival of a proliferating, quiescent, or hypoxic cancer cell comprising contacting the proliferating, quiescent, or hypoxic cancer cell with an effective amount of an agent that inhibits a protein involved in lactate transport or enzymatic conversion in the proliferating, quiescent, or hypoxic cancer cell.

In an aspect, the disclosure relates to a method of blocking or inhibiting the survival or growth of a neoplastic cell comprising contacting the neoplastic cell with an agent that inhibits lactate clearance, and wherein the neoplastic cell comprises at least one of: (a) increased lactate concentration or (b) decreased glucose concentration relative to a cell from normal tissue.

In an aspect, the disclosure relates to methods of treating or preventing cancer in a subject who has, or is at risk of developing, a cancer that comprises a proliferating, quiescent, or hypoxic cancer cell, wherein the method comprises administering to the subject an agent in an amount effective to inhibit a lactate catabolic pathway in the proliferating, quiescent, or hypoxic cancer cell.

In a further aspect, the disclosure relates to a method of inducing intracellular acidification in a proliferating, quiescent, or hypoxic cancer cell comprising contacting the proliferating, quiescent, or hypoxic cancer cell with an agent in an amount effective to inhibit at least one of a transport protein or a catabolic and/or metabolic enzyme involved in lactate clearance in the proliferating, quiescent, or hypoxic cancer cell.

In another aspect, the disclosure provides a method of inducing reductive stress in a proliferating, quiescent, or hypoxic cancer cell comprising contacting the proliferating, quiescent, or hypoxic cancer cell with an agent in an amount effective to inhibit at least one of a lactate transport protein and a lactate catabolic or metabolic protein in the proliferating, quiescent, or hypoxic cancer cell.

In an aspect, the disclosure provides a method of inhibiting glycolysis in a proliferating, quiescent, or hypoxic cancer cell comprising contacting the proliferating, quiescent, or hypoxic cancer cell with an agent in an amount effective to inhibit at least one of a lactate transport protein and a lactate catabolic or metabolic protein in the proliferating, quiescent, or hypoxic cancer cell.

Aspects of the disclosure relate to methods of adjuvant therapy. In some of these aspects, the disclosure provides a method of sensitizing a proliferating, quiescent, or hypoxic cancer cell to a therapeutic regimen comprising contacting the proliferating, quiescent, or hypoxic cancer cell with an agent in an amount effective to inhibit at least one of a lactate transport protein and a lactate catabolic or metabolic protein in the proliferating, quiescent, or hypoxic cancer cell.

Aspects of the disclosure relate to assays and/or methods for screening candidate compounds for activity as lactate clearance inhibitors.

Other aspects and embodiments are encompassed by the disclosure and will become apparent in light of the following description and accompanying drawings.

### BRIEF DESCRIPTION OF THE DRAWINGS

FIG. 1 illustrates passive lactate uptake in R3230 cells. (A) Cells incubated under normal (6%) and hypoxic (0.5%) pO2 levels, at various glucose and lactate concentrations. (B) ¹H NMR spectra of perchloric acid (PCA) extracts of cells incubated with ¹³C-labelled lactate. (C) ¹³C NMR spectra of perchloric acid (PCA) extracts of cells incubated with ¹³C-labelled lactate. (D) Autoradiograph of a section from a R3230 tumor grown a nude mouse after exposure to ¹⁴C-labelled lactate. (E) Immunohistochemical staining of MCT-1 lactate transporter in R3230 tumor cells (upper panel) and rat skeletal muscle (lower panel).

FIG. 2 depicts the effect of lactate on R3230 cell survivability and toxicity. (A) Effect of extraneous lactate concentrations on R3230 survival (by analysis of cell attachment), varying glucose concentrations (18 hr incubation; 6% O₂; 5% CO₂; 37° C.). (B) Effect of extraneous lactate concentrations on R3230 survival (colony forming fraction) in presence and absence of glucose (24 hr incubation; 21% O₂). (C) Effect of exogenous lactate on glucose consumption under glucopenic conditions (18 hr incubation; 6% O₂; 5% CO₂; 37° C.). (D) Effect of exogenous lactate concentration on glucose consumption under glucopenic conditions (18 hr incubation; 6% O₂; 5% CO₂; 37° C.). (E) Effect of exogenous lactate concentration on cellular pH. (F) Effect of Na-lactate concentration on cellular NAD/NADH ratio (expressed as light absorbance ratio, 260/340 nm).

FIG. 3 depicts catabolism of ¹³C methyl-labelled lactate. (A) Fluorescence immunostain of MCT-1 in skeletal muscle (top) and R3230 tumour (bottom) of a Fischer 344 rat. Green: MCT-1, Blue: exogenous perfusion marker (Hoechst 33342). MCT-1 is ubiquitously expressed in R3230 cells, showing a membranous and cytoplasmic stain. (B) Left panel, shows ¹³C-labelled glutamate and alanine in cell extracts is apparent after 4 hr incubation, and cleared at 24 hr, and that CHC is able to inhibit lactate clearance; right panel shows that while lactate, glutamate, and alanine are all present in the growth medium at 24 hr, the addition of CHC effectively inhibited catabolism of lactate to glutamate and alanine (C) Illustrates effect of lactate concentration on ability of CHC to clear lactate via catabolic pathways. (D) Cell survivability as a function of lactate concentration in the presence of varying amounts of CHC (0, 1, and 5 mM). (E) Lactate conversion to glutamate and alanine in R3230 tumors grown in vivo, measured by ¹³C NMR and quantified with respect to total protein content. Time points (baseline, 15, 30, and 60 min) refer to start of infusion.

FIG. 4 depicts appearance of lactate and its metabolic products glutamate and alanine using ¹³C NMR spectra, in various human cancer types including human glioblastoma (GBM-245), human head/neck cancer (FaDu), and breast cancer (MCF-7 and MDAMB-231).

FIG. 5 depicts a schematic diagram of cellular lactate clearance via glutamate and alanine production. Lactate enters the cell primarily via proton-coupled co-transport (e.g., by monocarboxylate transporters, MCTs). After conversion to pyruvate (pyr), it can be converted to alanine, via alanine-glutamate-transaminase (ALAT) that uses glutamate as an amino group donor, possibly sourced from mitrochondrial glutamate generated from lactate. Alternatively lactate (or pyruvate generated from lactate) enters the mitochondria, proceeds through the TCA cycle and is converted to glutamate via glutamate dehydrogenase. Both alanine and glutamate are exported from the cell.

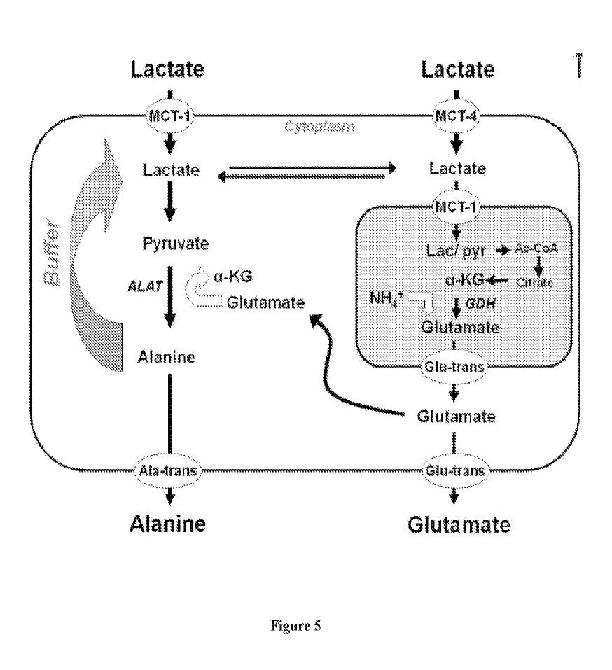

### DETAILED DESCRIPTION

It will be understood that the various aspects and embodiments described herein are merely intended to provide illustration and do not serve to limit the scope of the claims. The disclosure relates to a number of alternative aspects and other embodiments and can be carried out in a variety of alternative and effective ways.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

The inventors have found that mammalian cells use a range of biochemical pathways to break down unwanted and/or excess environmental lactate, and excrete its catabolites. As described herein, blockade of these mechanisms such as, for example, by inhibiting intracellular pyruvate transport, leads to cancer cell death under high lactate concentrations. The targeting and modulation of the lactate detoxification pathway can provide for one of the few treatment options that would be effective against both proliferating and quiescent cancer cells. This is even more relevant, since the quiescent fraction of a solid tumor generally incorporates chronically hypoxic cancer cells, which are considered the most treatment-resistant cell type of a solid tumor. Moreover, hypoxic cancer cells are under chronically elevated reductive stress, caused by the limited availability of oxygen as a terminal electron acceptor. Lactate-induced toxicity escalates this reductive stress. At the same time, because of the diffusion geometry of solid tumors, chronically hypoxic cells are generally exposed to higher lactate levels than non-hypoxic cancer cells. As detailed in the following description and illustrative Examples, pharmacological inactivation of lactate clearance mechanisms can target chronically hypoxic cancer. The approach taken herein that targets a biochemical protection mechanism that enables cancer cells to survive in the tumor microenvironment is not typical, and to the knowledge of the inventors, is not found in the literature.

Thus, in a general sense the disclosure relates to active agents and therapeutic methods that are effective in the treatment of cancers, such as solid tumors, that are associated with proliferating, quiescent, and hypoxic cancer cells. The inventors have found that some cancer cell lines not only produce, but also consume exogenous lactate, e.g., in catabolic production of other biomolecules and/or as a fuel for respiration. For example, the inventors have shown that the R3230Ac tumor can produce and accumulate lactate to concentrations that exceed 20 mM in vivo. As detailed in the Examples, the disclosure demonstrates that R3230 cells not only produce lactate, but are able to take up exogenous lactate. Moderate concentrations of lactate (e.g., ˜2.5 mM) can promote survival of aerobic cancer cells under low glucose conditions, however continuous exposure to elevated concentrations of lactate (e.g., >5 mM) is typically toxic to the cells. Cancer cells however, such as the R3230 cells used for illustrative purposes herein, have an effective clearing mechanism that allows the cell to remove exogenous lactate that has passively entered the cell, from the intracellular space within 24 h of exposure to elevated lactate concentrations. As shown in the Examples, lactate can be cleared by a number of cellular catabolic pathways, including pathways that convert lactate to the amino acids alanine and/or glutamate, which are subsequently released into the extracellular medium. However, data from the NMR spectra of lactate catabolic products indicates that additional catabolic products (catabolites) exist. Accordingly, agents and methods that inhibit one or more members of a lactate catabolic pathway and/or a lactate exporter protein are effective to inhibit lactate clearance and can induce or increase the cytotoxic effect of high lactate concentrations in proliferating, quiescent, or hypoxic cancer cells, and thereby reduce the survival of cancer cells.

In an aspect, the disclosure relates to a method of inhibiting the survival of a proliferating, quiescent, or hypoxic cancer cell comprising contacting the proliferating, quiescent, or hypoxic cancer cell with an agent in an amount effective to inhibit a lactate catabolic pathway in the proliferating, quiescent, or hypoxic cancer cell.

In some embodiments the agent inhibits one or more targets that are associated with the catabolic conversion of lactate to alanine such as, for example, an alanine export protein and an alanine transaminase. Alanine export proteins can include, for example, PAT-1 (Gene map locus: 5q31-q33, GenBank accession BC136438), sodium dependent and sodium independent membrane transporters, and transporters that are sensitive and insensitive to 2-(methylamino)isobutyric acid. Alanine transaminases can include, for example, a cytoplasmic (soluble) glutamic-pyruvate transaminase (GPT1) and a mitochondrial glutamic-pyruvate transaminase (GPT2).

In some embodiments the agent inhibits one or more targets that are associated with the catabolic conversion of lactate to glutamate such as, for example, a glutamate transport protein and a glutamate dehydrogenase. Glutamate transport proteins can include, for example, proteins encoded by the following genes: SLC1A3 (GenBank NM 019225.1), EAAT2 (GenBank UO3505), SLC1A2 (GenBank NM_{—}017215.2), EAAT3 (GenBank U39555), SLC1A1 (GenBank BC033040), EAAT4 (GenBank U18244.1), SLC1A6 (GenBank BC028721), EAAT5 (GenBank U76362), SLC1A7 (GenBank BC017242), VGLUT1 (GenBank U07609), SLC17A7 (GenBank NM_{—}020309), VGLUT2 (GenBank AF271235), SLC17A6 (GenBank NM_{—}020346), VGLUT3 (GenBank AL157942), SLC17A8 (GenBank NG_{—}021175), and SLC17A5 (GenBank AF244577) (encoding sialin). Glutamate dehydrogenases can include, for example, glutamate dehydrogenase 1 (GLUD1, also termed GDH, GLDH, mitochondrial GDH), or glutamate dehydrogenase 2 (GLUD2), and the like.

In some embodiments the agent inhibits one or more targets that are associated with the catabolic conversion of lactate, via pyruvate, into oxaloacetate, malate, and aspartate, and with the export of these catabolites from the cytoplasm. Potential targets include pyruvate carboxylase, malate dehydrogenase, and aspartate aminotransferase, and inhibitors of these catabolites. Examples of inhibitors of these targets include phenylacetate, carboxyphosphate, carbamoyl phosphate, methylmalonyl-CoA, oxamate, and oxyacetate.

In some embodiments the agent inhibits one or more targets that are associated with the catabolic conversion of lactate to a product other than alanine and glutamate, which can be identified by any number of techniques including, for example, ¹³C based NMR methods as described herein. Monitoring and/or quantifying the appearance and/or disappearance of the peaks appearing at characteristic chemical shifts (e.g., integral function of resonances at particular ppm, relative to a reference) can be used to evaluate the efficacy of the particular agent being evaluated or the course of therapeutic treatment. Similar methods for monitoring and quantifying the appearance and/or disappearance of the peaks appearing at characteristic chemical shifts for alanine and glutamate can also be used in assays for screening candidate compounds that inhibit lactate clearance, or for an assay to evaluate the efficacy of treatment.

In another aspect, the disclosure relates to a method of inhibiting the survival of a proliferating, quiescent, or hypoxic cancer cell comprising contacting the proliferating, quiescent, or hypoxic cancer cell with an effective amount of an agent that inhibits a lactate or pyruvate transporter protein in the proliferating, quiescent, or hypoxic cancer cell. In some embodiments, the protein comprises MCT-4 or MCT-1. In some embodiments, the agent comprises AR-C 117977, N-Phenylmaleimide, 2-oxo-4-methylpentanoate, Phenyl-pyruvate, GW604714X, GW450863X, or CHC (α-cyanohydroxycinnamate).

In embodiments of the methods disclosed herein, the proliferating, quiescent, or hypoxic cancer cell comprises a solid tumor. In further embodiments, the solid tumor comprises a melanoma, carcinoma, hepatoblastoma, neuroblastoma, osteosarcoma, retinoblastoma, endocrine tumor, or desmoid tumor. In some embodiments, solid tumor comprises cancer of the breast, cervix, ovary, colon, rectum, anus, stomach, kidney, larynx, liver, lung, brain, head, neck, prostate, testicle, or bladder.

In an aspect, the disclosure relates to a method of inhibiting the proliferation of a neoplastic cell comprising contacting the neoplastic cell with an agent that inhibits lactate clearance, and wherein the neoplastic cell comprises at least one of: (a) a detectable lactate concentration or (b) decreased glucose concentration relative to a cell from normal tissue, or both. In some embodiments the neoplastic cell comprises an amount of lactate be an amount that occurs naturally within the particular cell or tissue type. In some embodiments the neoplastic cell comprises a lactate concentration greater than 2.0 mM (e.g., about 2.1 mM, about 2.2 mM, about 2.3 mM, about 2.4 mM, about 2.5 mM, about 2.6 mM, about 2.7 mM, about 2.8 mM, about 2.9 mM, about 3.0 mM, about 3.5 mM, about 4.0 mM, about 4.5 mM, about 5.0 mM, about 5.5 mM, about 6.0 mM, about 6.5 mM, about 7.0 mM, about 7.5 mM, about 8.0 mM, about 8.5 mM, about 9.0 mM, about 9.5 mM, about 10.0 mM, about 15.0 mM, about 20.0 mM, about 25.0 mM, about 30.0 mM, about 35.0 mM, about 40.0 mM, and so on), up to about 80 mM. In some embodiments, the neoplastic cell comprises a glucose concentration less than about 5 mM (e.g., less than 5.0 mM, less than 4.5 mM, less than 4.0 mM, less than 3.9 mM, less than 3.8 mM, less than 3.7 mM, less than 3.6 mM, less than 3.5 mM, less than 3.4 mM, less than 3.3 mM, less than 3.2 mM, less than 3.1 mM, less than 3.0 mM, less than 2.9 mM, less than 2.8 mM, less than 2.7 mM, less than 2.6 mM, less than 2.5 mM, less than 2.4 mM, less than 2.3 mM, less than 2.2 mM, less than 2.1 mM, less than 2.0 mM, less than 1.9 mM, less than 1.8 mM, less than 1.7 mM, less than 1.6 mM, less than 1.5 mM, less than 1.4 mM, less than 1.3 mM, less than 1.2 mM, less than 1.1 mM, less than 1.0 mM, and so on) to 0 mM. In some embodiments the neoplastic cell comprises a combination of the above lactate and glucose concentrations of about 1.0 mM to about 5.0 mM or, in further embodiments about 1.5 mM (on the order of micromoles per gram of tissue) and less.

In an aspect, the disclosure relates to methods of treating or preventing cancer in a subject who has, or is at risk of developing, a cancer that comprises a proliferating, quiescent, or hypoxic cancer cell, wherein the method comprises administering to the subject an agent in an amount effective to inhibit a lactate catabolic pathway in the proliferating, quiescent, or hypoxic cancer cell. In some embodiments the subject has a cancer that comprises a solid tumor. In further embodiments, the subject has a solid tumor that comprises a melanoma, carcinoma, hepatoblastoma, neuroblastoma, osteosarcoma, retinoblastoma, endocrine tumor, or desmoid tumor. In some embodiments, the subject has a solid tumor that comprises cancer of the breast, cervix, ovary, colon, rectum, anus, stomach, kidney, larynx, liver, lung, brain, head, neck, prostate, testicle, or bladder.

In embodiments of the disclosed aspects relating to methods of treating a subject, the subject can be an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. In certain embodiments, the subject is a mammal. In further embodiments, the mammal is a human.

“Inhibiting”, “blocking”, or “reducing” when used herein in relation to inhibiting or reducing the survival of a cell, such as a proliferating, quiescent, or hypoxic cancer cell, neoplastic cell, solid tumor, and the like, refers to reducing, inhibiting, or preventing the survival, growth or differentiation of a cell such as, for example, a cancer cell. In some embodiments “reducing” or “inhibiting” cell survival, growth or differentiation includes killing a cell, and thus encompasses decreasing the number of cancer cells and/or reducing the size of a tumor. The term “treatment”, as used herein in the context of treating a condition, pertains generally to treatment and therapy of a subject, in which a desired therapeutic effect is achieved. For example, treatment may ameliorate the condition or may inhibit the progress of the condition (e.g., reduce the rate of progress or halt the rate of progress). Thus, the terms “treating” and “treatment” when used with reference to a disease or a subject in need of treatment includes, but is not limited to, halting or slowing of disease progression, remission of disease, prophylaxis of symptoms, reduction in disease and/or symptom severity, or reduction in disease length as compared to an untreated subject. In embodiments, the methods of treatment can abate one or more clinical indications of the particular disease being treated. Certain embodiments relating to methods of treating a disease or condition associated with cancer (e.g., solid cancers) and comprise administration of therapeutically effective amounts of an inhibitor of lactate clearance as well as pharmaceutical compositions thereof. In embodiments, the method of treatment can relate to any method that prevents further progression of the disease and/or symptoms, slows or reduces the further progression of the disease and/or symptoms, or reverses the disease and/or clinical symptoms associated with the disease. The term “preventing” when used in connection with methods of preventing cancer in a subject at risk of developing a cancer refers to reducing the likelihood that cancer will occur in a subject as well as reducing the likelihood that cancer will recur in a subject who has previously been afflicted with a cancer.

A “quiescent” cell as used herein relates to a cell that can be at any point in the cell cycle and considered to be resting (i.e., not actively dividing).

A “proliferating” cell as used herein relates to a cell that is in an active process associated with cell division (i.e., in the process of dividing, and not resting (“quiescent”)) in any point in the cell cycle.

“Hypoxic” cells as used herein relates to one or more cells that are exposed, transiently or permanently, to an oxygen partial pressure (pO2) that is lower than the typical pO2 in cells in tissue that is considered as normal or healthy. Hypoxic cells can include, for example, cells with reduced or no access to vasculature, such as in a solid tumor.

In some aspects the methods relate to inhibiting or reducing lactate clearance in a cancer cell. In embodiments, lactate clearance is associated with at least one of lactate catabolism, lactate transport (e.g., lactate export), glutamate release, and alanine release. As described herein, some embodiments relating to lactate catabolism can comprise one or more of the production of alanine, glutamate, as well as other products that are associated with lactate catabolism, and combinations thereof.

As used herein the term “contacting a cell” is used to mean contacting a cell directly or indirectly in vitro, ex vivo, or in vivo (i.e. within a subject, such as a mammal, including humans, mice, rats, rabbits, cats, and dogs). Contacting may occur as a result of administration to a subject. Contacting encompasses administration to a cell, tissue, mammal, patient, or human. Further, contacting a cell includes adding an agent to a cell culture. Other suitable methods may include introducing or administering an agent to a cell, tissue, mammal, or patient using appropriate procedures and routes of administration as disclosed herein or otherwise known in the art.

In embodiments, the methods described herein include administering to a subject an effective amount of an inhibitor of lactate clearance in combination with a second treatment. “Co-administered” or “co-administration” refer to simultaneous or sequential administration. A compound may be administered before, concurrently with, or after administration of another compound. In such embodiments, the second treatment can include such non-limiting examples as surgery, radiation, and chemotherapy. In further embodiments, the method comprises co-administration of an effective amount of an inhibitor of lactate clearance and a second agent that acts as a bioreductive prodrug or hypoxic cytotoxin such as, for example, AQ4N (Novacea, Inc.), PR-104 (Proacta, Inc.), TH-302 (Threshold Pharmaceuticals). Suitably the co-administration does not include an agent that can effectively increase oxygen supply to a hypoxic cell, as the inhibitor of lactate clearance can be highly effective against cancer cells in a quiescent state, which is usually associated with shortage of oxygen, nutrients, and/or growth factors.

Aspects of the disclosure relate to methods of adjuvant therapy. In some of these aspects, the disclosure provides a method of sensitizing a proliferating, quiescent, or hypoxic cancer cell to a therapeutic regimen comprising contacting the proliferating, quiescent, or hypoxic cancer cell with an agent in an amount effective to inhibit at least one of a lactate export protein and a lactate catabolic protein in the proliferating, quiescent, or hypoxic cancer cell. In some embodiments, the method of treatment can be used an adjuvant therapy (i.e., additional treatment) such as, for example, when an inhibitor of lactate clearance, or pharmaceutical compositions thereof, are administered after surgery or other treatments (e.g., radiation, hormone therapy, or chemotherapy). Accordingly, in such embodiments, the method of adjuvant therapy encompasses administering an inhibitor of lactate clearance to a subject following a primary or initial treatment, and can be administered either alone or in combination with one or more other adjuvant treatments, including, for example surgery, radiation therapy, or systemic therapy (e.g., chemotherapy, immunotherapy, hormone therapy, or biological response modifiers). Those of skill in the art will be able to use statistical evidence to assess the risk of disease relapse before deciding on the specific adjuvant therapy. The aim of adjuvant treatment is to improve disease-specific and overall survival. Because the treatment is essentially for a risk, rather than for provable disease, it is accepted that a proportion of patients who receive adjuvant therapy will already have been effectively treated or cured by their primary surgery. Adjuvant therapy is often given following surgery for many types of cancer including, for example, colon cancer, rectal cancer, anal cancer, brain cancer, head and neck cancer, lung cancer, pancreatic cancer, breast cancer, prostate cancer, and some gynecological cancers.

Some embodiments of the method relate to neoadjuvant therapy, which is administered prior to a primary treatment. Effective neoadjuvant therapy is commonly characterized by a reduction in the number of cancer cells (e.g., size of the tumor) so as to facilitate more effective primary treatment such as, for example, surgery. Some embodiments provide a method of neoadjuvant therapy comprising administering an amount of an inhibitor of lactate clearance that is effective to sensitize the cancer cells to one or more therapeutic regimen (e.g., chemotherapy or radiation therapy).

The term “cancer” refers to or describes the physiological condition in mammals that is typically characterized by unregulated cell growth. In some embodiments, cancer can comprise a solid tumor or cancerous mass. Solid tumors include, but are not limited to, melanoma, carcinoma, blastoma (e.g., hepatoblastoma, neuroblastoma, retinoblastoma), glioblastoma, sarcoma (e.g., osteosarcoma), endocrine tumors, desmoid tumors, and germ cell tumors. Solid tumor may comprise cancer associated with certain organs including, but not limited to, the breast, cervix, ovary, colon, rectum, anus, kidney, larynx, liver, brain, head, neck, lung, prostate, testicle, and bladder. Some non-limiting examples of cancers that fall within these broad categories include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, melanoma, brain cancer (e.g., giant cell glioblastoma and gliosarcoma), as well as head and neck cancers, and associated metastases.

The term “cancer” also encompasses cell proliferative disorders which are associated with some degree of abnormal cell proliferation, and includes tumors. “Tumor” as used herein, refers to any neoplasm or neoplastic cell growth and proliferation, whether malignant or benign, and all pre-cancerous and cancerous cells and tissues.

“Administration” or “administering” refers to delivery of the compounds by any appropriate route to achieve the desired effect. Thus, administration of an effective amount of an agent that inhibits of lactate clearance, suitably lactate catabolism or lactate export, may be carried out by any means known in the art including, but not limited to intraperitoneal, parenteral, intravenous, intramuscular, subcutaneous, or transcutaneous injection; oral, sublingual, transdermal, topical, buccal, rectal, nasopharyngeal or transmucosal absorption; implants; or inhalation. Such administration encompasses the administration of an inhibitor of lactate clearance formulated as a pharmaceutical composition. Delivery (administration route) also includes targeted delivery wherein the inhibitor of lactate clearance is only active in a targeted region of the body (e.g., in a particular organ or tissue, or localized to the solid tumor mass), as well as sustained release formulations in which the inhibitor compound is released over a period of time in a controlled manner. Sustained release formulations and methods for targeted delivery are known in the art and include, for example, use of liposomes, drug loaded biodegradable microspheres, drug-polymer conjugates, drug-specific binding agent conjugates and the like. Pharmaceutically acceptable carriers are well known to those of skill in the art. Determination of particular pharmaceutical formulations and therapeutically effective amounts and dosing regimen for a given treatment is within the ability of one of skill in the art taking into consideration, for example, patient age, weight, sex, ethnicity, organ (e.g., liver and kidney) function, the extent of desired treatment, the stage and severity of the disease and associated symptoms, and the tolerance of the patient for the treatment.

In a further aspect, the disclosure provides a method of inducing intracellular acidification in a proliferating, quiescent, or hypoxic cancer cell comprising contacting the proliferating, quiescent, or hypoxic cancer cell with an agent in an amount effective to inhibit at least one of a lactate export protein and a lactate catabolic protein in the proliferating, quiescent, or hypoxic cancer cell. In some embodiments the method is effective to inhibit the conversion of lactate to one or more of its catabolic products such as, for example, alanine or glutamate. In embodiments the proliferating, quiescent, or hypoxic cancer cell is contacted with an amount of agent that is effective to inhibit the export of lactate from the cell, and not effective to inhibit passive or active cellular uptake of lactate. In some embodiments, the agent induces a decrease in intracellular pH of greater than 0.05 pH units to about 0.3 pH units.

In another aspect, the disclosure provides a method of inducing reductive stress in a proliferating, quiescent, or hypoxic cancer cell comprising contacting the proliferating, quiescent, or hypoxic cancer cell with an agent in an amount effective to inhibit at least one of a lactate export protein and a lactate catabolic protein in the proliferating, quiescent, or hypoxic cancer cell. In embodiments the method induces the conversion of NAD+ to NADH and H+. In further embodiments the amount of the agent is effective to reduce the intracellular concentration of free NAD+ by about 10% to about 50% relative to the amount of NADH. In some embodiments, the method inhibits cellular dehydrogenases that are dependent on NAD+. In other embodiments the method induces reductive stress that is effective to inhibit one or more members of cellular survival pathways such as, for example, HIF-2α.

In an aspect, the disclosure provides a method of inhibiting glycolysis in a proliferating, quiescent, or hypoxic cancer cell comprising contacting the proliferating, quiescent, or hypoxic cancer cell with an agent in an amount effective to inhibit at least one of a lactate export protein and a lactate catabolic protein in the proliferating, quiescent, or hypoxic cancer cell. In some embodiments, the amount of agent added is effective to induce or provide an intracellular concentration of lactate of about 2.5 mM to about 80 mM (e.g., about 2.5 mM, about 2.6 mM, about 2.7 mM, about 2.8 mM, about 2.9 mM, about 3.0 mM, about 3.5 mM, about 4.0 mM, about 4.5 mM, about 5.0 mM, about 5.5 mM, about 6.0 mM, about 6.5 mM, about 7.0 mM, about 7.5 mM, about 8.0 mM, about 8.5 mM, about 9.0 mM, about 9.5 mM, about 10.0 mM, about 15.0 mM, about 20.0 mM, about 25.0 mM, about 30.0 mM, about 35.0 mM, about 40.0 mM, and so on), up to about 80 mM.

### Active Agents and Compounds

In some embodiments of the above disclosed methods comprise at least one agent that is effective to inhibit lactate clearance (e.g., lactate catabolism, lactate export, alanine transport, glutamate transport, pyruvate transport, etc.). Compounds can be tested for such activity using the methods and assays described herein, as well as any method known in the art. In some embodiments the agent can comprise L-cycloserine, aminooxyacetic acid, L-serine O-sulphate, chlorpromazine, desipramine, imipramine, amitriptyline, chloroquine, kainite, isoprenaline, salfinamide, and lamotigine, AR-C 117977, N-Phenylmaleimide, 2-oxo-4-methylpentanoate, Phenyl-pyruvate, GW604714X, GW450863X, or CHC.

### Compositions and Formulations

Aspects of the disclosure relate to compositions and formulations, including pharmaceutical compositions and formulations, that comprise an effective amount of an agent as described herein (e.g., an agent that can inhibit the proliferation of a proliferating, quiescent, or hypoxic cancer cell, inhibit lactate clearance, inhibit lactate transport, inhibit a lactate catabolic pathway, induce intracellular acidification, induce reductive stress, inhibit glycolysis, and the like). Such compositions and formulations comprise an effective amount of an agent in combination with a carrier, vehicle, excipient, or diluent, including pharmaceutically acceptable carriers, vehicles, excipients, and diluents. An “effective amount” relates to a quantity of an agent that high enough to provide a significant positive modification of the subject's condition to be treated, and is suitably low enough to avoid serious side effects (at a reasonable benefit/risk ratio). Carriers, vehicles, excipients, and diluents can be one or more compatible substances that are suitable for administration to a mammal such as, for example, solid or liquid fillers, diluents, hydrotopes, surface-active agents, and encapsulating substances. “Compatible” means that the components of the composition are capable of being commingled with the inhibitor, and with each other, in a manner such that there is no interaction which would substantially reduce the efficacy of the composition under ordinary use situations. Carriers, vehicles, excipients, and diluents are suitably of sufficiently high purity and sufficiently low toxicity to render them suitable for administration to the mammal being treated. The carrier, vehicle, excipient, or diluent can be inert, or it can possess pharmaceutical benefits and/or aesthetic benefits, or both. Suitable carriers, vehicles, excipients, and diluents are known in the art and can be found in standard pharmaceutical texts, for example, Remington's Pharmaceutical Sciences, 18th edition, Mack Publishing Company, Easton, Pa., 1990, incorporated herein by reference.

### Assays and Screening Methods

Aspects of the disclosure relate to assays and methods that can be used to identify a candidate compound having activity that interferes with lactate clearance (e.g., catabolism, transport, etc.). Such a compound can be termed a “lactate clearance inhibitor.” The illustrative methods that are discussed in the Examples have applicability and use in such assays and screening methods. Thus, in some aspects, methods of identifying a lactate clearance inhibitor are provided. In certain embodiments, the method comprises contacting a cell with a candidate compound agent and using an appropriate assay to detect and/or quantify the amount of lactate clearance in response to the candidate compound. In certain embodiments, when the compound decreases the rate of lactate clearance, the agent is considered to be lactate clearance inhibitor. In certain embodiments, when the agent increases the amount of lactate clearance detected, the agent is considered to be a lactate clearance inhibitor. Embodiments of such an assay or method can comprise contacting a cell such as, for example, a cancer cell that exhibits at least one activity associated with a lactate clearance mechanism (e.g., lactate transport, or lactate catabolism) with an amount of the candidate compound under conditions that would allow for normal lactate clearance, and monitoring the effect the candidate compound has on the cellular rate and/or quantity of lactate clearance, relative to a control cell. Such assays and methods can employ any known reagent or technique including, but not limited to, fluorescence, radioisotope tracers, specific binding assays, NMR methods, cell survival assays, colormetric assays, and the like. In embodiments, the candidate compound is identified as lactate clearance inhibitor when it provides a detectable decrease in lactate clearance in a cell when contacted with an amount of the compound, relative to a control cell that is not contacted with the compound. Suitably, an inhibitor will decrease at least one pathway of lactate clearance (measured by a decrease in the rate of clearance or decrease in the amount of clearance (e.g., processed lactate)) by about 10%, 20%, 30%, 40%, 50%, 60%, 70%, 80%, or 90% or more relative to a control. Such compounds can be evaluated as cancer therapeutics and used in the methods described herein.

The following examples provide further description of certain aspects and embodiments that fall within the scope of the broader disclosure. As the examples are provided merely to illustrate some aspects of the description, they should not be viewed as limiting the scope of the appended claims.

### EXAMPLES

### Materials and Methods

Tissue culture and measurement of metabolic rates: The R3230 rat mammary carcinoma was originally derived from a spontaneous mammary carcinoma of the Fischer 344 rat (Hilf et al, 1965; Lindberg et al, 1996). Cells were exposed to 6% or 0.5% oxygen (Vaupel et al, 1991) at 37° C., and 0.5% CO₂ in glucose- and serum-free DMEM, while dishes were placed on a slow-moving rocker. Cells were counted and media samples were collected at the beginning and end of the experiment, and analyzed for glucose and lactate content using a CMA microdialysis analyzer (CMA, Sweden).

¹H-NMR and ¹³C-NMR: R3230 cells were grown to 60% confluency. Cells were washed and exposed to DMEM with varying concentrations of ¹³C-methyl labeled L-lactate solution (Sigma, St. Louis, Mo.), without serum and glucose. Cells were washed, mounted with 60% perchloric acid, scraped down, and centrifuged at 8,000 G for 5 min; PBS (20% vol/vol) and 98% deuterated water (10% vol/vol) (Sigma, St. Louis, Mo.) were added to supernatants to a total of 600 uL for NMR. For medium sample analysis, 10% vol/vol deuterated water was added to the medium before NMR analysis. NMR spectra were recorded on a Varian 500 MHz VNMRS spectrometer equipped with a Dell Optiplex 755 data system and a 5 mm Varian inverse probe. ¹H NMR spectra were obtained with a spectral width (SW) of 5.5 kHz, a 67 pulse flip angle (6 us), a 6.4 s acquisition time (AT), a 2 sec water presaturation pulse and relaxation delay (RD), and digitized using 64000 points to obtain a digital resolution of 0.172 Hz per point. ¹H-decoupled ¹³C spectra were recorded at 125 MHz with a 32051.3 Hz SW, a 60 pulse flip angle (6.5 us), a 2.0 s AT, a 0.7 s RD and digitized into 130,000 points to yield a digital resolution of 0.493 Hz per point.

pHi measurements: Cells were grown on glass coverslides and incubated with BCECF-AM (Invitrogen) at 5 μM for 20 min, and imaged on an inverted fluorescence microscope, with excitation at 440 and 490 nm, emission 535 nm. Ratiometric pH calibration was done in mixture ratios of buffers containing 110 mM K₂HPO₄ or 135 mM KH₂PO₄, and 20 mM NaCl, respectively. Nigericin was added at 10 μM (Franck et al, 1996) and pH expressed as ratios of grayscale values from stacks of calibration images taken from the nigericin-treated cells, imaged at 440 and 490 nm (Franck et al, 1996).

Measurement of redox changes: Cells were trypsinized, washed, and brought to 5×10⁶/mL in PBS. 1 mL of cell suspension was scanned for optical density in a cuvette at 260 nm (NAD⁺) and 340 nm (NADH+H⁺). Various concentrations of Na-lactate in PBS were added at 20 μL/mL.

Clonogenic assay: Cultured cells were incubated for 18 h at 37° C. in serum free DMEM, with varying concentrations of Na-lactate. Cells were washed and returned to normal growth medium. 14 days later, colonies were stained with crystal violet and counted.

Autoradiography: Athymic nude mice transplanted with R3230Ac tumours were infused with two μCi of ¹⁴C-lactate (GE, Pittsburg Pa.). The hypoxia marker, pimonidazole, was injected at 60 mg/kg i.p. Hoechst-33342 was administered intravenously as a perfusion marker dye (10 mg/mL, 0.05 mL), and tumours were harvested and snap-frozen. Tumours were cryosectioned for autoradiography, Hoechst, and pimonidazole staining Sections were exposed to storage phosphor screen (Packard Bioscience, Downers Grove, Ill.) for 3 weeks for autoradiography.

Uptake of ¹³C -labeled lactate in vivo by NMR: Fisher-344 rats, with tumour chunks being transplanted from donor animals were allowed to grow to 1-2 cm in diameter. Rats were fasted for four hours, then anesthetized with isofluorane and placed on a heating pad. All procedures involving animals were performed in compliance with the guidelines of the Duke University Institutional Animal Care Committee. ¹³C-lactate (100 mM in 1 mL) was infused into the femoral vein using an infusion pump at a 0.1 mL/min and tumours were collected and snap-frozen in liquid. Frozen tissues were pulverized under liquid nitrogen, with perchloric acid added (2 mL in 0.9 M), and homogenized at 4° C. in a Dounce homogenizer. NMR measurements were carried out as described above.

Uptake kinetics of ¹⁴C-lactate in vivo: Kinetics of ¹⁴C-lactate or ¹⁴C-glucose uptake were studied using a novel implantable fiber optic radiation detection probe provided by Sicel Technologies Inc., Durham, N.C. Time uptake curves from tumor, subcutaneous, and blood compartments were analyzed using a 3-compartment model from which were derived parameters related to the transfer coefficients in and out of the tumor and blood clearance (see Supplemental methods for detail). The apparatus detected beta radiation from ¹⁴C, which is proportional to ¹⁴ C-lactate or ¹⁴C-glucose concentration. Probes were inserted into tumors of anesthetized rats (Isoflurane as above) and subcutaneous tissue as a control. Each probe was calibrated using ¹⁴C-glucose solutions of known concentration (μCi/mL) immediately before the experiment. After a stabilization period of 50 min, 50 μCi of ¹⁴C-lactate or ¹⁴C-glucose was infused intravenously. Data were recorded in two-minute intervals over 3 h. Baseline (background, dark current values) were subtracted from each time point.

Normalization to the calibration solution was done to control for variations in probe performance and length of probe used in different tumors. The relationship between photons detected and concentration (μCi/mL) was linear, based on experiments done by the company. Data were graphed as the change in μCi/mL over time, and normalized to dose/body weight (μCi/g) to obtain a standardized uptake value (SUV).

In separate animals, blood samples (300 μL-500 μL) were taken at 0, 4, 7, 10, 15, 25, 30, 60, 100, and 160 minutes from the arterial cannula following ¹⁴C-radioisotope infusion. Samples were centrifuged, and plasma collected and weighed. Plasma activities were measured in a liquid scintillation counter (Packard Tri-Carb 1500, Perkin Elmer Life and Analytical Sciences Inc., Boston Mass.) and isotope concentrations (μCi/g) were calculated. Blood activity data were normalized to dose/body weight. Time uptake curves from tumor, subcutaneous, and blood compartments were analyzed using a 3-compartmental model.

The equations defining tracer kinetics were the following:

dCp/dt=−(k1+k4+k0)Cp+k2Ct+k5Cs (1)

dCi/dt=k1Cp−k2Ci (2)

dCs/dt=k4Cp−k5Cs (3)

Ct=VbCp+Ci (4)

Cp, Ci, and Cs were the tracer concentration in central blood compartment, tumor cells, and subcutaneous tissue, respectively. Ct was the measured compound concentration in tumor. Vb was the vascular volume fraction. Due to the nature of the measurement, the tumor probe collected photon counts from both tumor cells and nearby vasculature. The last equation described vascular contribution to the overall photon counts in the tumor tissue. Ki was the transfer constant between each compartment. The averaged isotope concentration in plasma was used to create an empirical blood curve to be used in the model. Experimental data were fitted to this compartmental model and parameters were estimated using SAMII software.

### Example 1

### Lactate Uptake in R3230 Cells

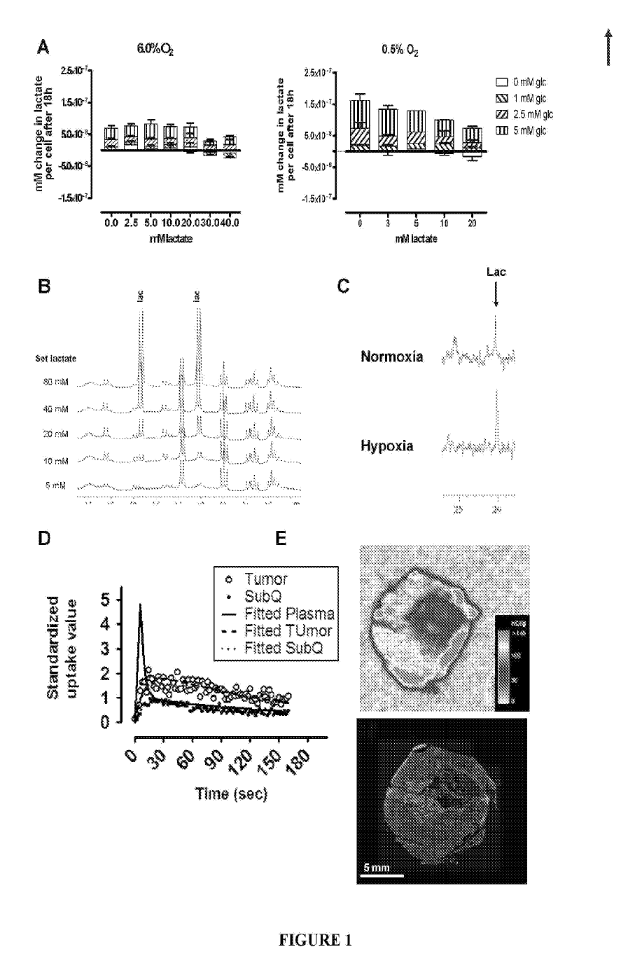

Consistent with the Pasteur effect, lactate production in R3230 cells was higher under hypoxia than normoxia, and lactate production roughly depended on glucose availability (FIG. 1A) (Krebs, 1972). Increasing external lactate concentration inhibited cellular production of lactate (FIG. 1A). Net lactate consumption was observed under normoxia when glucose was ≦1 mM and lactate ≧30 mM. In the absence of glucose, net lactate consumption was observed even under hypoxia (FIG. 1A, 20 mM lactate). Lactate uptake in R3230 was concentration dependent, reflected by ¹H NMR spectra on PCA extracts (FIG. 1B, 4 h incubation, normoxia, no glucose). ¹³C NMR on PCA extracts of R3230 cells incubated with 40 mM ¹³C-methyl labeled lactate for 12 h confirmed lactate uptake under both normoxia and hypoxia (FIG. 1C).

Kinetic uptake experiments and autoradiography after infusion of ¹⁴C-labeled lactate to R3230 tumour-bearing rats demonstrated that lactate was taken up by this tumour in vivo (FIG. 1D). Infused lactate was retained in the tumour, even after clearance from the plasma, and interstitium (FIG. 1D).

Rate constants (Table 1) were obtained by fitting from the experimental data. Comparison of the in vs. outflow rates of lactate and glucose revealed that lactate was taken up at ˜5 times higher rates than glucose. The transfer rate constant for lactate into tumour being 5-fold higher than that for glucose strongly suggested that active metabolism of this compound was occurring in tumours.

TABLE 1
Rate constants for ¹⁴C-labeled lactate and glucose in R3230 Ac

| Metabolite | Parameter | Explanation | Value | SD |
|---|---|---|---|---|
| Glucose | K1 | Transfer rate into tumor | 0.038 | 0.0077 |
| | K2 | Transfer rate out of tumor | 0.049 | 0.0057 |
| | K4 | Transfer rate into SQ | 0.0016 | 9.36E−05 |
| | K5 | Transfer rate out of SQ | 0.066 | 0.0075 |
| Lactate | K1 | Transfer rate into tumor | 0.23805 | 5.48E−02 |
| | K2 | Transfer rate out of tumor | 0.06182 | 9.60E−03 |
| | K4 | Transfer rate into SQ | 0.00144 | 1.82E−04 |
| | K5 | Transfer rate out of SQ | 0.07439 | 9.10E−03 |

Comparison of ¹⁴C-autoradiography with hypoxia/perfusion stain (pimonidazole=orange/ Hoechst 33342=blue) on slices from R3230 tumours grown as xenografts in mice demonstrated that lactate uptake occurred in both normoxic and hypoxic cells, although it was lower in hypoxic cells (FIG. 1E). Without being limited to any particular mechanism, this may have been due to competition between exogenous ¹⁴C-lactate with non-labeled lactate arising from glycolysis (Schroeder et al, 2005).

### Example 2

### Effect of External Lactate on R3230 Cells

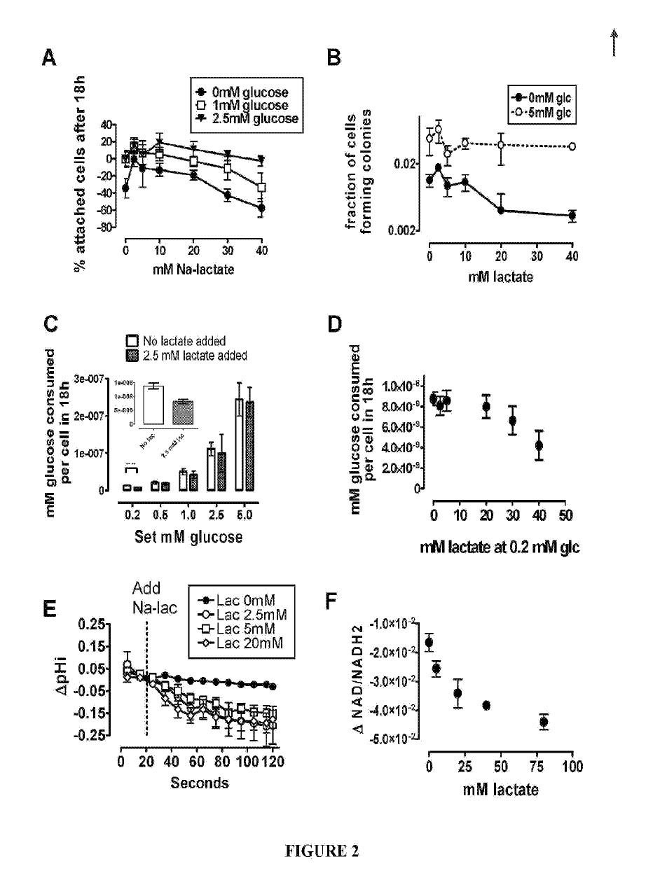

Under glucose deprivation, Na-lactate concentrations of >2.5 mM led to decreased survival of R3230 cells, as measured by counts of attached cells (FIG. 2A). Colony formation assays confirmed the toxic effect of lactate under glucose and serum deprivation (FIG. 2B). However, lactate concentrations of 2.5 mM rendered a survival advantage under glucose starvation over any other lactate concentration (FIG. 2A, B).

Under very low glucose concentrations of 0.2 mM (a concentration often seen in human tumors and this tumor model (Schroeder et al, 2005)), lactate inhibited glucose consumption by 41% (FIG. 2C). Escalated extracellular lactate concentrations inhibited glucose consumption in a concentration-dependent manner (FIG. 2D). Lactate induced concentration-dependent reduction of intracellular pH, as measured by the fluorescent dye BCECF-AM (FIG. 2E). Lactate also caused acute reductive stress, indicated by a concentration-dependent drop NAD⁺/ NADH₂⁺ within 2 minutes after (isovolumic) addition (FIG. 2F).

### Example 3

### R3230 Cells Convert Lactate into Alanine and Glutamate

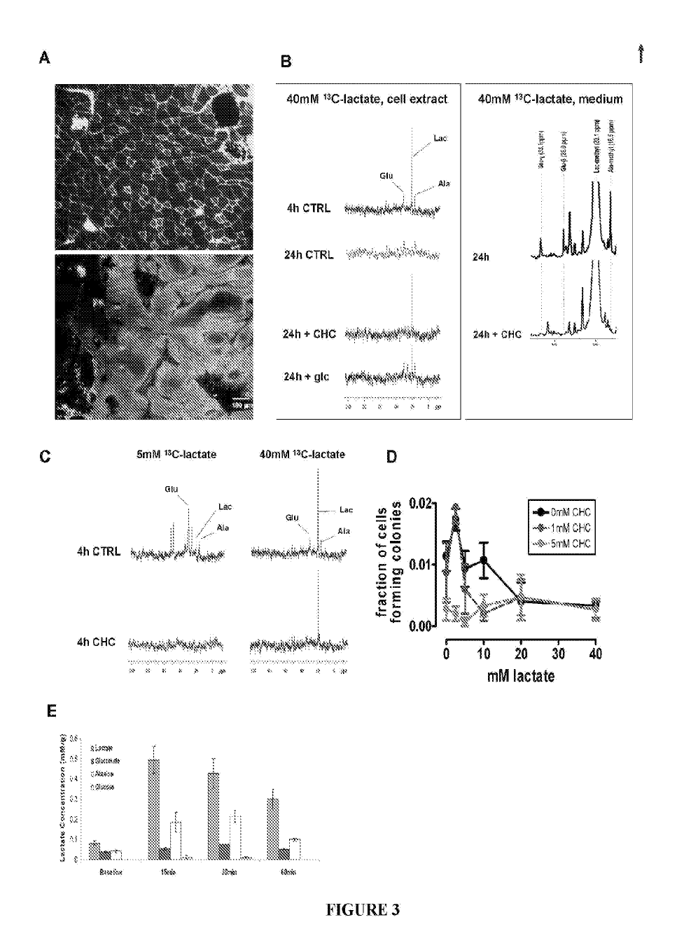

Lactate uptake may be dependent on the monocarboxylate transporter MCT-1 (Brooks, 2002). Immunostaining in R3230 tumours demonstrated the presence of MCT-1 in R3230 cells (FIG. 3A Bottom panel). The expression of MCT-1 in rat skeletal muscle demonstrated membranous localization of MCT-1 in lactate consuming tissues (oxidative muscle fibers) (Bonen et al, 2000) (FIG. 3A upper panel). ¹³C NMR of washed cell extracts after incubation of R3230 cells in culture with 40 mM lactate (glucose/ serum free) confirmed that lactate was taken up after 4 h (FIG. 3B, left panel). Alanine and glutamate appeared in the medium after 4 h (FIG. 3B,C). All traces of ¹³C lactate disappeared from the intracellular space after 24 h (FIG. 3B). However, ¹³C NMR of media supernatant demonstrated that labeled alanine and glutamate accumulate in the medium during 24 h incubation (FIG. 3B). Addition of glucose inhibited lactate catabolism (FIG. 3B). Blockade of MCT-1 by 5 mM α-cyano-4-hydroxycinnamate (CHC) inhibited breakdown of lactate and conversion into alanine and glutamate within 24 h (FIG. 3B, left panel), and consequently, their accumulation in the culture media (FIG. 3B). Whereas 5 mM CHC effectively inhibited lactate uptake under low lactate conditions (5 mM), lactate uptake persisted with lactate concentrations of 40 mM (FIG. 3C). Low concentrations of CHC selectively reduced R3230 clonogenic survival at high ambient lactate (>5mM), but not low lactate (0-5 mM). Higher concentrations of CHC were cytotoxic, independent of ambient lactate (FIG. 3C).

¹³C-NMR on tissue homogenates of R3230 tumours grown in the flanks of Fisher 344 rats showed that ¹³C alanine and ¹³C glutamate concentrations rise after infusion of lactate into the host, providing evidence that lactate catabolism occured in vivo. The slight increase in glucose after 30 min reflected conversion of lactate into glucose via gluconeogenesis in the liver (FIG. 3E).

### Example 4

### Catabolic Pathway of Lactate in Human Cancer Cell Lines

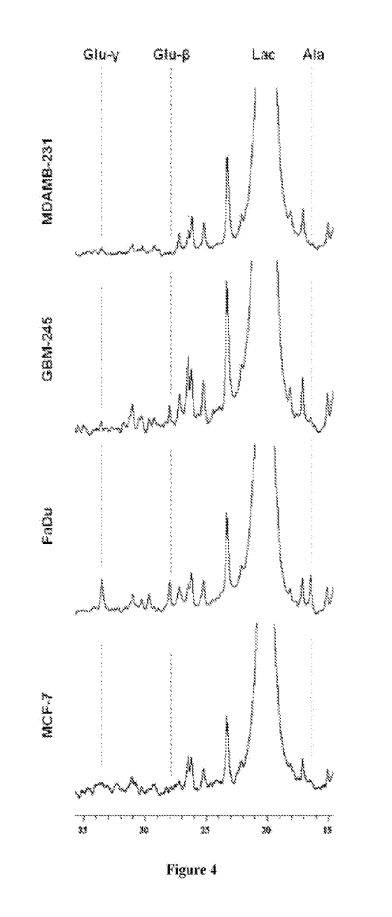

Human laryngeal cancer cells (FaDu) and glioblastoma lines (GBM-245) accumulated ¹³C alanine and/or ¹³C glutamate from ¹³C-lactate after 24 h exposure to 40 mM ¹³C-lactate (no glucose and serum) in a manner similar to that of the R3230Ac tumour. In contrast, two human breast cancer cell lines (MDAMB-231 and MCF-7) did not produce these catabolites, although multiple yet unidentified peaks were observed (FIG. 4). Control experiments with ¹²C labeled lactate, and spiking with the substrate of interest confirmed substrate identity and incorporation of the label.

We claim:

1. A method of inhibiting the survival or growth of a proliferating, quiescent, or hypoxic cancer cell comprising contacting the proliferating, quiescent, or hypoxic cancer cell with an agent in an amount effective to inhibit a lactate catabolic pathway in the proliferating, quiescent, or hypoxic cancer cell.

2. The method of 1 wherein the lactate catabolic pathway is associated with alanine production.

3. The method of 2 wherein the method inhibits at least one protein selected from an alanine export protein and an alanine transaminase.

4. The method of claim 3, wherein the alanine export protein is PAT-1, sodium dependent and sodium independent membrane transporters, and transporters that are sensitive and insensitive to 2-(methylamino)isobutyric acid.

5. The method of claim 3, wherein the alanine transaminase is cytoplasmic glutamic-pyruvate transaminase (GPT1) or mitochondrial glutamic-pyruvate transaminase (GPT2).

6. The method of claim 1 wherein the lactate catabolic pathway is associated with glutamate production.

7. The method of 6 wherein the method inhibits at least one protein selected from a glutamate transport protein and a glutamate dehydrogenase.

8. The method of claim 7, wherein the glutamate transport protein is SLC1A3, EAAT2, SLC1A2, EAAT3, SLC1A1, EAAT4, SLC1A6, EAAT5, SLC1A7, VGLUT1, SLC17A7, VGLUT2, SLC17A6, VGLUT3, SLC17A8, and SLC17A5.

9. The method of 7 wherein the glutamate dehydrogenase is glutamate dehydrogenase 1 (GLUD1) or glutamate dehydrogenase 2 (GLUD2).

10. The method of claim 1, wherein the agent comprises at least one of L-cycloserine, aminooxyacetic acid, L-serine O-sulphate, α-cyanohydroxycinnamate, chlorpromazine, desipramine, imipramine, amitriptyline, chloroquine, kainite, isoprenaline, salfinamide, and lamotigine AR-C117977, N-Phenylmaleimide, 2-oxo-4-methylpentanoate, Phenyl-pyruvate, GW604714X, or GW450863X.

11. A method of inhibiting the survival of a proliferating, quiescent, or hypoxic cancer cell comprising contacting the proliferating, quiescent, or hypoxic cancer cell with an effective amount of an agent that inhibits lactate detoxification in the proliferating, quiescent, or hypoxic cancer cell.

12. The method of claim 11, wherein the lactate transporter protein comprises MCT-4 or MCT-1.

13. The method of claim 11, wherein the agent comprises AR-C117977, N-Phenylmaleimide, 2-oxo-4-methylpentanoate, Phenyl-pyruvate, GW604714X, GW450863X, or CHC.

14. The method of any one of claims 1-13, wherein the proliferating, quiescent, or hypoxic cancer cell comprises a solid tumor.

15. The method of claim 14, wherein the solid tumor comprises a melanoma, carcinoma, hepatoblastoma, neuroblastoma, osteosarcoma, retinoblastoma, endocrine tumor, or desmoid tumor.

16. The method claim 14, wherein the solid tumor comprises cancer of the breast, cervix, ovary, colon, rectum, anus, stomach, kidney, larynx, liver, lung, brain, head, neck, prostate, testicle, or bladder.

17. The method of any one of claims 1-16, wherein the method further comprising contacting the proliferating, quiescent, or hypoxic cancer cell with a second anti-cancer agent.

18. The method of claim 17, wherein the second anti-cancer agent comprises a chemotherapeutic agent or radiation therapy.

19. A method of inhibiting the survival of a neoplastic cell comprising contacting the neoplastic cell with an agent that inhibits lactate clearance, and wherein the neoplastic cell comprises at least one of an increased lactate concentration or a decreased glucose concentration relative to a cell from normal tissue.

20. A method of treating or preventing cancer in a subject who has, or is at risk of developing, a cancer that comprises a proliferating, quiescent, or hypoxic cancer cell, wherein the method comprises administering to the subject an agent in an amount effective to inhibit a lactate catabolic pathway in the proliferating, quiescent, or hypoxic cancer cell.

21. The method of claim 20, wherein the cancer comprises a solid tumor.

22. The method of claim 21, wherein the solid tumor comprises a melanoma, carcinoma, hepatoblastoma, neuroblastoma, osteosarcoma, retinoblastoma, endocrine tumor, or desmoid tumor.

23. The method claim 21, wherein the solid tumor comprises cancer of the breast, cervix, ovary, colon, rectum, anus, stomach, kidney, larynx, liver, lung, brain, head, neck, prostate, testicle, or bladder.

24. The method of any of claims 18-23, wherein the agent comprises at least one of α-cyano-4-hydroxycinnamate (CHC), L-cycloserine, aminooxyacetic acid, L-serine O-sulphate, A-cyanohydroxycinnamate, AR-C117977, chlorpromazine, desipramine, imipramine, amitriptyline, cloroquine, kainite, isoprenaline, salfinamide, and lamotigine.

25. The method of claim 24, wherein the agent is administered in combination with a second anti-cancer agent.

26. The method of claim 25, wherein the second anti-cancer agent comprises a chemotherapeutic agent or radiation therapy.

27. A method of inducing intracellular acidification in a proliferating, quiescent, or hypoxic cancer cell comprising contacting the proliferating, quiescent, or hypoxic cancer cell with an agent in an amount effective to inhibit at least one of a lactate export protein and a lactate catabolic protein in the proliferating, quiescent, or hypoxic cancer cell.

28. A method of inducing reductive stress in a proliferating, quiescent, or hypoxic cancer cell comprising contacting the proliferating, quiescent, or hypoxic cancer cell with an agent in an amount effective to inhibit at least one of a lactate export protein and a lactate catabolic protein in the proliferating, quiescent, or hypoxic cancer cell.

29. A method of inhibiting glycolysis in a proliferating, quiescent, or hypoxic cancer cell comprising contacting the proliferating, quiescent, or hypoxic cancer cell with an agent in an amount effective to inhibit at least one of a lactate export protein and a lactate catabolic protein in the proliferating, quiescent, or hypoxic cancer cell.

30. A method of sensitizing a proliferating, quiescent, or hypoxic cancer cell to a therapeutic regimen comprising contacting the proliferating, quiescent, or hypoxic cancer cell with an agent in an amount effective to inhibit at least one of a lactate export protein and a lactate catabolic protein in the proliferating, quiescent, or hypoxic cancer cell.
